(19) United States
(12) Patent Application Publication
(10) Pub. No.: US 2006/0247505 A1
(43) Pub. Date: Nov. 2, 2006

(54) Wireless sensor system

(76) Inventor: Waqaas Ahmed Siddiqui, Toronto (CA)

Correspondence Address: OGILVY RENAULT LLP, 1981 MCGILL COLLEGE AVENUE, SUITE 1600, MONTREAL, QC, H3A2Y3, CA

(21) Appl. No.: 11/411,827

(22) Filed: Apr. 27, 2006

Related U.S. Application Data
(60) Provisional application No. 60/675,456, filed on Apr. 28, 2005.

(51) Int. Cl. A61B 5/00

(52) U.S. Cl. 600/300

(57) ABSTRACT

A wireless sensor system is provided, utilising any number of sensor modules, any number of mobile communication devices, and a server. The sensor modules have sensors for sampling data, and wirelessly transmit data to their corresponding mobile communication device, and receive transmission instructions from the same. The mobile communication devices transmit the sampled data to the server, wherein the data is processed using analysis software, and provided through interface software to a network in a useable form. The mobile communication devices display the sensor data in useable form by processing it with its own software module, allowing a user of the sensor module to view the sampled data. Other users may access the sensor data over the network to monitor the same. if an emergency situation is detected, the user of the sensor module can be directly contacted through their mobile communication device, and emergency personnel can be dispatched.

### FIELD OF THE INVENTION

The present invention relates generally to data acquisition systems and more particularly, to wireless sensor systems.

### DESCRIPTION OF THE PRIOR ART

In certain environments, it may be desired to periodically monitor particular physical parameters, to enable an observer to take cognisance of any anomalous situations that may arise, and subsequently take the appropriate action to remedy these situations. Such monitoring is particularly desirable in the medical industry.

For medical purposes, some patients may require periodic or continuous monitoring of vital signs, such as but not limited to, heart rate, blood pressure, blood-oxygen level, and pace maker statistics. If monitoring these vital signs is particularly crucial, e.g., following a threatening ailment, surgery etc., the patients are usually required to either remain under the supervision of a doctor or nurse at a hospital or to have expensive equipment installed at their home. In either situation, to achieve adequate monitoring capabilities, the patient would need to choose whether to remain at the hospital, or to have the expensive and complicated equipment set up at their home, in order to enjoy the comforts of recuperating in their own home.

If equipment is set up at a patient's home, the patient would either need to monitor the equipment (and thus the vital signs) themselves or have medical personnel take the responsibility of doing the monitoring. Both situations can be costly and invasive to the patient's everyday life. Moreover, self-monitoring by a patient can pose potential risks, since the patient would need to be instructed how to detect emergency situations, and if an emergency did arise, it may be difficult for the patient to contact emergency personnel to remedy the situation.

In a different scenario, a patient may require continuous monitoring of a vital sign on a day-to-day basis. Such a situation arises if a patient is, e.g., at risk for a future heart attack, and their pace maker statistics require monitoring to determine if any treatment or hospitalisation is needed. This type of monitoring is useful for preventative diagnoses. in this situation, it is unfeasible to have complicated equipment installed in the patient's home, undergo daily hospital visits or have constant supervision. Even if equipment is set up at a patient's home, it would be an impediment to the patient's daily routine, and still requires the patient to remain in the vicinity of the equipment to monitor the vital signs. Therefore, it can be difficult for the patient to proceed with their daily activities, whilst safely monitoring the necessary vital sign(s).

Patient monitoring systems have been implemented in order to have vital signs monitored by a practitioner, who is situated at a remote location. Such a system is described in U.S. Patent Application No. 2004/0236189 A1 to Hawthorne et al., published on Nov. 25, 2004. The system described by Hawthorne provides a portable bio-information unit, which acquires sensor data from a subject. The bio-information unit wirelessly sends the acquired data to a modem which is connected to a network. The network gathers the data and provides the data over the Internet to a treatment provider who can monitor the acquired data.

Hawthorne's system allows a patient to freely move around, while the bio-information unit gathers sensor data transmits the data, through the modem, to the network, where it can be analysed. However, the patient would need to be in the proximity of the modem to allow the sensor data to be transmitted to the Internet. Therefore, the patient is restricted to movement within an area having a suitable modem, which is capable of transmitting the data. Such a system is suitable for a patient that remains in their home. However, the patient would not be allowed to move out of the “reach” of the modem for prolonged periods. If the patient is being monitored from a remote location, periods of inactivity may trigger a false alarm (e.g. the patient is out of the reach of the modem and has not communicated to the treatment provider for a certain period of time).

Moreover, Hawthorne does not provide a convenient way for the treatment provider to contact the patient, should an emergency arise. The patient also becomes removed from the monitoring process, and therefore, would need to rely on the treatment provider to detect anomalous situations, and to take the necessary action to remedy the situation.

The above-described drawbacks are also applicable to other data acquisition systems, such as environmental monitoring systems, and seismic monitoring systems.

It is therefore an object of the present invention, to provide a sensor system that obviates or mitigates at least one of the above-described disadvantages.

### SUMMARY OF THE INVENTION

The present invention provides a portable sensor system, which allows sensor data to be monitored from any remote location. Both a user of the sensor system and an external entity may monitor the sensor data, and a communication link between the user and emergency personnel can be provided.

In one aspect, the present invention provides a wireless sensor system comprising at least one sensor module, at least one mobile communication device, a server, and a software program. The sensor module communicates with the mobile communication device to transmit data sampled by a sensor, and the mobile communication device communicates with the server to upload the data thereto. The data sent to the server is processed by the software program, provided over a network, and is accessible by a monitoring entity through the network, wherein the monitoring entity may monitor the data in order to detect an emergency.

In another aspect, the present invention provides a method for acquiring and monitoring sensor data comprising the steps of sampling data from at least one sensor controlled by a sensor module; sending the sampled data to a mobile communication device; the mobile communication device uploading the data to a server connected to a software program; the software program processing the data into a useable form; and the software program providing the data in its useable form to a monitoring entity over a network connected to the software program; wherein the data may be monitored by the monitoring entity, enabling the monitoring entity to initiate a response to a detected emergency.

### BRIEF DESCRIPTION OF THE DRAWINGS

The features of the invention will become more apparent in the following detailed description in which reference is made to the appended drawings wherein:

FIG. 1 is a schematic view of a wireless sensor system.

FIG. 2 is a schematic view of the sensor module shown in FIG. 1.

FIG. 3 is a schematic view of the mobile communication device shown in FIG. 1.

FIG. 4 is a flowchart showing a wireless data acquisition process.

FIG. 5 shows a login interface window.

FIG. 6 shows a data entry interface.

FIG. 7 shows a PC interface console.

FIG. 8 shows a graphical sensor output interface.

FIG. 9 shows a further graphical sensor output interface.

FIG. 10 shows a server connection console.

FIG. 11 shows an interface console,

FIG. 12 provides a representation of a communication from a sensor module to a mobile device.

FIG. 13 provides a representation of a communication from a mobile device to a sensor module.

FIG. 14 shows a mobile device to sensor module data frame.

FIG. 15 shows a sensor module to mobile device data frame.

FIG. 16 shows an acknowledgement frame.

### DETAILED DESCRIPTION OF THE INVENTION

The preferred embodiment of the present invention will be described, by way of example only, as implemented for wireless monitoring in a medical application.

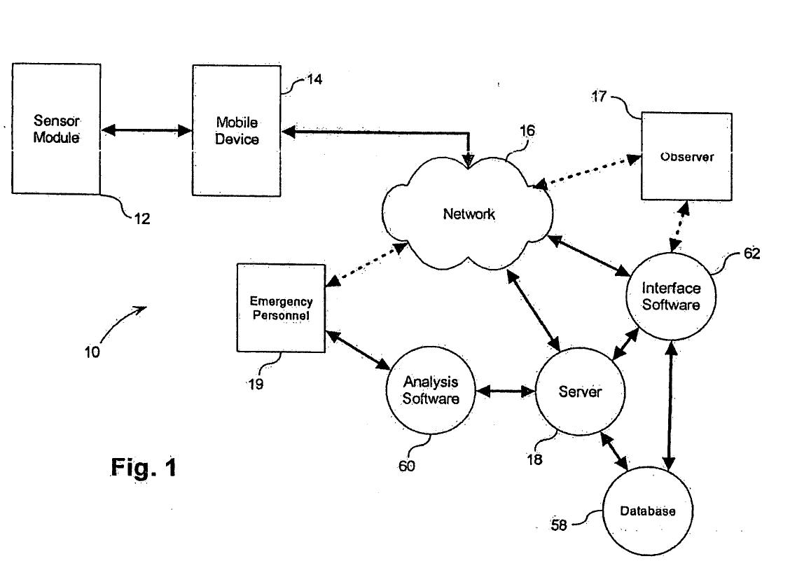

Referring therefore to FIG. 1, a wireless sensor system is generally denoted by numeral 10. The system 10 generally comprises a sensor module 12, which is worn by a human, and a mobile communication device 14 carried by the human. The device 14 communicates over a network 16 with a server 18. The server 18 interacts with analysis software 60 and interface software 62, and can access a database 58. Data provided by the server 18 and the database 58, can be accessed by an observer 17 through the interface software 62; and emergency personnel 19 can monitor data provided by the server 18 using the analysis software 60.

The network 16 may be any type of network, such as the Internet, or an internal Intranet. For the purpose of this description, we will assume that the network 16 is the Internet. The device 14 communicates with the sensor module 12 and the network 16 through wireless radio frequency (RF) communication protocols. The server 18, interface software 62, and analysis software 60 may be connected to the network 16 in any suitable manner, e.g., through a landline or wireless connection. The emergency personnel 19 may be connected to the system 10 in any suitable manner, such as through a telephone connection, Internet connection etc. The emergency personnel 19 can interact directly with the analysis software 60 through, e.g., a phone line, or by way of an alert sent through the network 16. The observer 17 is any user that has access to the server 18 and database 58, through the interface software 62. The observer 17 may be a treatment provider, or other entity in charge of monitoring the system. Although one observer 17 is shown in FIG. 1, it will be appreciated that any number of observers may access the server 18 and database 58 either directly using the interface software 62 or through the network 16, by possessing the proper authority to do so.

The sensor module 12 is positioned at the point of data acquisition in the embodiment described, the sensor module 12 is worn by a human, and the data acquired will comprise vital signs, such as a heart rate, through one of an array of sensors. The system 10 may support several sensor modules 12, or several mobile devices 14, depending on the application. A single device 14 may collect data from several sensor modules 12, or each sensor module 12 may communicate with its own device 14. The system's modularity encourages expandability and flexibility, to accommodate any number of sensors, sensor modules 12 or mobile communication devices 14 as needed or desired.

The mobile device 14 can be any suitable device, whether it be custom built or a pre-existing device. A suitable device is one that is portable, capable of 2-way wireless data communication, and that permits interaction with the user through a visual or auditory interface. For the purposes of this description, the device 14 will be a pre-existing device, such as a cellphone or personal digital assistant PDA), having known capabilities such as wireless transmission and reception, and visual and auditory functionality.

The server 18 can be any server, as long as a connection can be made with the network 16, and as long as interactions can be made with the database 58, interface software 62, and analysis software 60. The emergency personnel 19 may comprise any entity which can respond to an anomalous situation detected by the analysis software 60. For the purpose of this description, we will assume that the emergency personnel 19 comprises medical emergency personnel. it shall be noted that contact with emergency personnel 19 is optional, depending on the application of the system 10.

The observer 17, for the purpose of this description, will be a treatment provider such as a doctor in charge of monitoring the data acquired by the sensor module 12. It will be appreciated that the observer 17, may alternatively be located with the server 18, interface software 62, and/or database 58, depending on the application of the system 10.

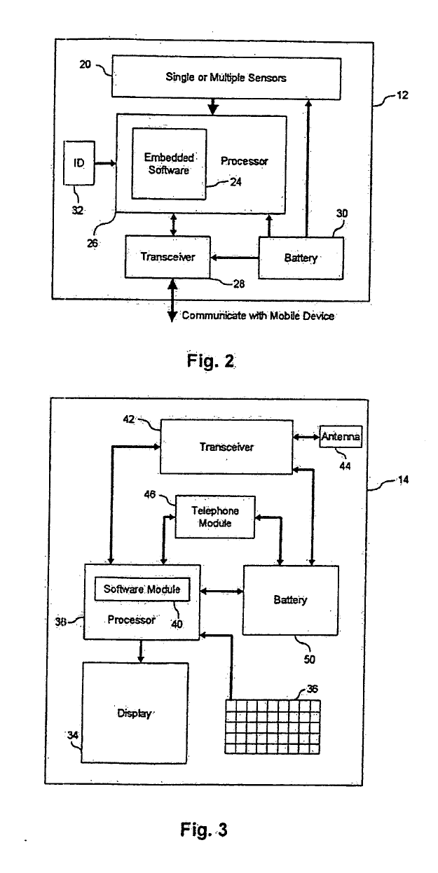

The sensor module 12 is shown in greater detail in FIG. 2. in this example, the sensor module 12 has one or more sensors 20. The sensors 20 can be hardwired to the sensor module 12, can be pluggable, or may even communicate wirelessly with the sensor module 12. The sensors 20 are connected to a Peripheral Interface Controller (PIC) microprocessor 26 having embedded software 24 for operation thereof. The microprocessor 26 has access to an identifier 32, which is unique to each particular module 12. The identifier 32 allows the server 18 to distinguish several sensor modules 12 from each other. The microprocessor 26 is connected to a transceiver 28, which communicates with the mobile device 14; and each of the processor 24, transceiver 28, and sensors 20 are powered by a battery 30.

The mobile device 14 is shown in greater detail in FIG. 3. The mobile device 14 has a display 34 and input device 36 which are externally visible to a user. In the case of a cell phone or PDA, the display 34 would be a screen, and the input device 36 would be a keypad. The display 34 and input device 36 are connected to a processor 38 having a software module 40. The software module 40 may be pre-loaded into the device 14, or may be downloaded by the user to enable the device 14 to be used with the system 10. The software module 40 coordinates the transfer of data from the sensor module 12 to the server 18, through the network 14, using the device 16. The processor 38 also communicates with a transceiver 42, which is connected to an antenna 44; and a telephone module 46. The device 14 also has a battery 50, which is preferably rechargeable battery. The telephone module 46 is an optional feature. The telephone module 46 is provided in this example since the system 10 is implemented using a cell phone or PDA. It will be appreciated that the transceiver 42 may also comprise a separate transmitter and receiver if desired.

The server 18 is used to receive and store data from the mobile device 14 sent through the network 16. Using the identifier 32, the server 18 can organize sensor data by correlating it with patient information. Preferably, the server 18 will only maintain a particular number of data samples for each sensor module 12 before overwriting out-of-date samples, to reduce memory consumption. The observer 17 can control this sample history size using the interface software 62. The server 18 is also responsible for governing the frequency of data collection, both from sensor module 12 to mobile device 14, and mobile device 14 to server 18. The server 18 is also responsible for maintaining and distributing updates/versions of the sensor module software 24 and the software module 40. The server 18 stores data that has been analysed using the analysis software 60 so that it may be accessible to the interface software 62. Parameters used by the analysis software 60 are also stored by the server 18.

The analysis software 60 is responsible for analyzing the data acquired by the sensor module 12, and correlating it to the patient in question. The software 60 is also responsible for the processing of the data to be stored on the server 18 so that the interface software 62 can access useable information. The analysis software 60 extracts sensor data from the server 18, and the server 18 provides the sensor parameters that need to be analysed from the sampled data. The analysis software 60 will also be responsible for detecting emergencies and taking the necessary steps to report such an emergency. It will be appreciated that although it is preferable for the analysis software 60 to detect emergencies in an automated fashion, the software 60 may also be used directly by the emergency personnel 19 depending on the particular application of the system 10.

The interface software 62 is responsible for providing an interface for the observer 17 to view sensor module data, control the analysis of the sensors 20, and for scheduling sampling rates for the mobile device 14 and the sensor module 12. The interface software 62 obtains inputs from the observer 17 with respect to refresh rates of the output as well as data collection frequency. These inputs from the observer 17 are stored by the server 18. The interface software 62, allows the observer 17 to view current sensor data and control the operation of the system 10. If authorized, the observer 17 may also view patient medical information using the interface software 62.

The database 58 is used to store data related to the patient, such as medical history, contact information, etc. The database 58 can acquire this information from either the observer 17, or a direct connection to patient servers at a particular institution, through the network 16. The database 58 is preferably a distinct module from the server 18 to minimize the load on the server 18, since the data will continue to accumulate as time progresses.

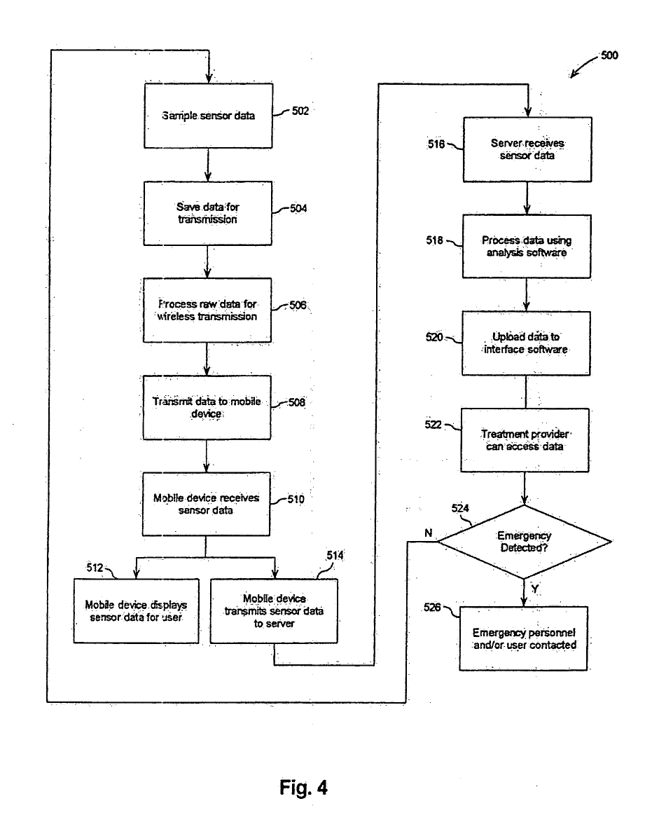

An typical wireless data acquisition process is shown in FIG. 4, and is generally denoted by numeral 500. An acquisition using a single sensor 20 will be described. The sensor module 12 samples sensor data from the sensor 20 at step 502. The sensor 20 reads data from the patient, and produces a signal that is sampled by the processor 26, using the software 24. At step 504, the microprocessor 26 saves the data for subsequent transmission to the device 14. The data that has been sampled and saved, is processed by the software 24 of the microprocessor 26 so that it conforms to a suitable wireless protocol at step 506. Details of appropriate wireless protocols will be explained in greater detail later.

The frequency of data transmission from the sensor module 12 to the mobile device 14, is controlled through messages sent by the mobile device 14. The software module 40 sends a message using the transceiver 42 and antenna 44 indicating the transmission schedule, to the sensor module 12, and then the data is transmitted back to the mobile device 14 by the sensor module 12, at step 508, using the transceiver 28. The data is received at step 510 by the mobile device's transceiver 42, through the antenna 44.

The software 40 will then process the sensor data. The mobile device 14 can optionally display the sensor data directly to the patient using the display 34 at step 512. The software 40, controlled by the processor 38, then commands the transceiver 42 to transmit the sensor data (that the device 14 has received from the sensor module 12) to the server 18 through its wireless connection to the network 16, established using the antenna 44, at step 514. The server 18 receives the sensor data from the mobile device 14, at step 516.

The analysis software 60 processes the sensor data at step 518, and correlates and stores it with patient information on the server 18. The server 18 then uploads the data to the interface software 62 at step 520, where it is available to the observer 17. The observer 17 (which in this case is a treatment provider) can view the processed sensor data provided by the interlace software 62 at step 522, either directly from the interface software 62 or through the network 16. Any connection made between the observer 17 and the server 18 and interface software 62 is preferably secure, requiring the observer 17 to have the proper authority to access the patient's information. The protocols implemented by the interface software 62 will be explained in greater detail later.

Once the sensor data is available for monitoring, the data is continuously monitored by the analysis software 60 at step 524. If an emergency is not detected, the sampling of sensor data continues. If an emergency is detected, the emergency personnel 19 and/or the patient are contacted at step 526. When an emergency occurs, the emergency personnel 19 can be dispatched directly from the analysis software 60 or the observer 17, depending on who is responsible. The emergency personnel 19 and/or observer 17 can then take the appropriate action to respond to the emergency.

The above generally describes the structure of the system and an exemplary implementation thereof, for monitoring sampled sensor data. The sensor data is sampled by the sensor module 12 and sent to the device 14 according to a suitable wireless protocol. The following describes a suitable wireless protocol to enable communication between the sensor module 12 and the device 14.

One suitable wireless protocol is the IEEE 802.15.4 standard. The 802.15.4 protocol is a Wireless Personal Area Network (WPAN) protocol that is geared towards simple, low-cost networks that require reliable data transfers in a relatively short range. The protocol implements the physical and Message Authentication Code (MAC) layers of the protocol stack.

A WPAN implementation would consist of a number of sensor modules 12 that are within each other's personal operating space (POS). All sensor modules 12 within a Personal Area Network (PAN) have a unique 64-bit address as part of their identifiers 32. This address is used for direct communication within the PAN.

A typical implementation uses a star topology consisting of a star network where the mobile device 14 is chosen as the coordinator of the network. In a star network, all sensor modules 12 communicate with the coordinator (i.e. the mobile device 14). Star networks operate independently from other star networks in the vicinity. This is achieved using the unique identifier 32. Once an identifier 32 has been chosen, the mobile device 14 can allow other sensor modules 12 to join its network.

The physical layer is responsible for the physical data service, and the physical management service that interfaces the other layers of the protocol stack. The main service of the physical layer, the physical data service, is responsible for the transmission and reception of protocol data units across the physical radio channel. The features of the physical layer are activation and deactivation of the radio transceivers 28 and 42, in order to minimize power consumption, low power detection, channel selection, clear channel assessment, and transmitting as well as receiving data packets. The radio has the option of operating in license free bands, such as 868-868.6 MHz in Europe, 902-928 MHz in North America, and 2400-2483.5 MHz worldwide.

The MAC sublayer is responsible for providing the MAC data service, and the MAC management service that is responsible for interfacing the MAC layer with other layers of the protocol stack. The MAC data service enables the transmission and reception of MAC protocol data units across the physical data service. The features of the MAC sublayer are mobile device management, channel access, frame validation, acknowledgement, association, and disassociation. The MAC layer can also be used to provide hooks for security implementations.

Typically, there are two ways for conducting data transfers within a PAN. The first type of data transfer is from the sensor modules 12 to the mobile device 14. The other type of data transfer is from the mobile device 14 to the sensor modules 12.

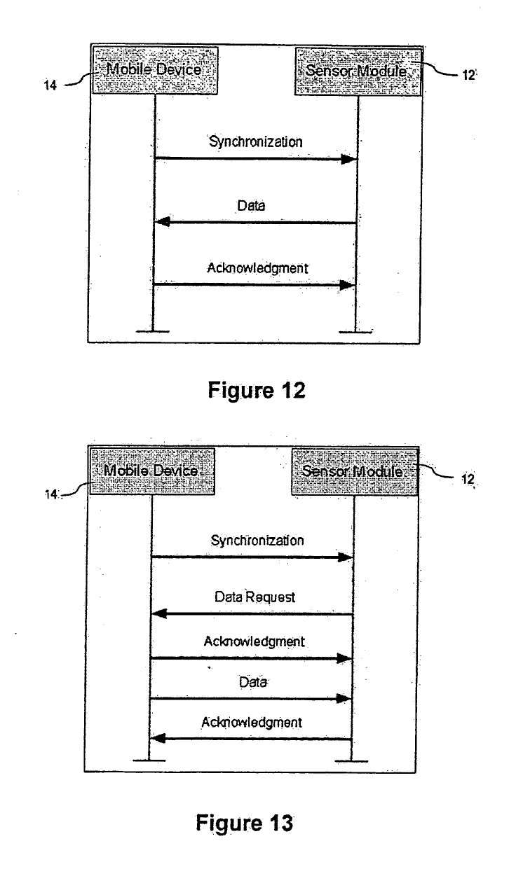

When a sensor module 12 wishes to transfer data to a mobile device 14, the first step is to listen for a synchronization message from the mobile device 14. When an indication of synchronization has been found, the sensor module 12 synchronizes with the mobile device 14. At an appropriate point in time, based on the particular sampling rate dictated by the server 18, the sensor module 12 transmits its data packet, using slotted Carrier Sense Multiple Access CA (CSMA-CA) to the mobile device 14. The mobile device 14 acknowledges the successful reception of data by transmitting an acknowledgement packet (ACK) back to the sensor module 12, as shown in FIG. 12.

When the mobile device 14 wishes to transfer data to the sensor module 12, it indicates this in the synchronization packet. The sensor module 12 processes the synchronization packet periodically. Upon discovering that a message is pending, the sensor module 12 transmits a MAC command requesting the data, using slotted CSMA-CA. The mobile device 14 acknowledges the successful data request by transmitting an ACK. The pending data from the mobile device is then sent to the sensor module 12, using slotted CSMA-CA. The sensor module 12 acknowledges the successful reception of data by transmitting an ACK. Upon receiving the ACK from the sensor module 12, the mobile device 14 removes the message from its list of pending messages. The above is generally shown in FIG. 13.

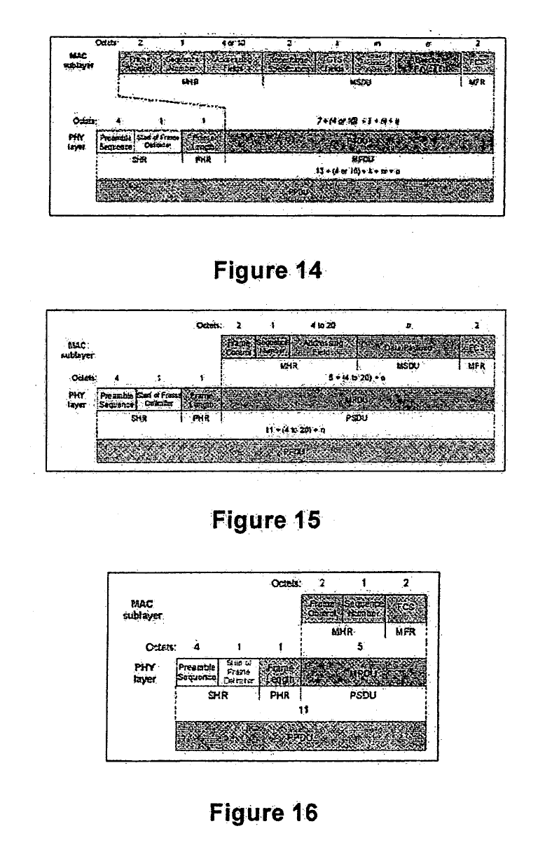

One type of data transmission in the PAN originates from the mobile device 14 and is sent to the sensor module 12. FIG. 14 shows the structure of the mobile device frame, which originates from the MAC sublayer. The MAC service data unit contains the synchronization, pending address specification, address list, and data fields. The MAC service data is prefixed with a MAC layer header and appended with a MAC layer footer. The MAC header contains the MAC layer frame control fields, PAN sequence number, and addressing information fields. The MAC footer contains a 16 bit frame error checking sequence. The MAC service data unit combined with the header and footer comprise the MAC protocol data unit. The MAC protocol data unit is sent to the physical layer for transmission. The MAC protocol data unit is used as the data packet in the physical service data unit. A synchronization header is prefixed to this in order to form the physical data packet.

The other data packet that can be sent in the PAN originates from the sensor module 12 and is sent to the mobile device 14. The only difference between this packet and the previous one is that instead of the data portion of the packet containing synchronization information and frame specifications, etc, it consists of just the raw data being sent from the sensor module 12 to the mobile device 14. FIG. 15 shows the packet breakdown of a data packet going from the sensor module 12 to the mobile device 14.

The MAC acknowledgement frame consists of the MAC header/footer. The MAC header is comprised of the MAC frame control and data sequence number. The MAC footer is comprised of a 16 bit frame error checking sequence. The MAC acknowledgement frame is then passed to the physical layer where the synchronization and physical headers are appended to the frame. The three of these comprise an acknowledgement frame. FIG. 16 shows the packet breakdown of an acknowledgement frame.

Synchronized PAN networks use a slotted CSMA-CA channel access mechanism, where the backoff slots are aligned with the start of the synchronization transmission. Every time a sensor module wants to transmit a data packet during the contention access period, it has to locate the boundary of the next backoff slot, and then wait for a random number of backoff slots. If the channel is detected to be busy, the sensor module shall wait for another random number of backoff slots before trying to access the channel again. If the channel is idle, the sensor module can begin transmitting in the next available backoff slot. Acknowledgement and synchronization frames are not sent using the CSMA-CA mechanism.

WPAN is flexible in many ways, including: allowing multiple topologies, different frequencies, any type of data can be transferred, permits synchronized or unsynchronized transmission schemes, allows optional acknowledgement, and variable data rates. These features make the 802.15.4 a robust protocol, due to its variable implementation capabilities. It is also a low power implementation, while providing adequate data rates. The protocol also provides reliable transmission through the use of ACKs.

The above protocol generally describes an adaptation of a known wireless protocol, namely the IEEE 802.15.4 protocol, and is shown for illustrative purposes only. It will be appreciated that many other suitable wireless protocols can be used depending on the application of the system 10, such as bluetooth or zigbee.

It shall be noted that non Radio Frequency (RF) communication protocols may also be used. For example, magnetic induction communication may be used between the sensor module 12 and the mobile device 14 Magnetic induction communication uses an induced magnetic field that creates a “bubble” around the operating space of the particular device. Since magnetic field does not interfere with other magnetic fields or electric fields, the amount of noise experienced can be greatly diminished. Thus, such communication would be less susceptible to interference. Moreover, magnetic induction communication is used over a short range and its drop off rate can be extremely fast. Outside of the “bubble” others cannot access information, which also provides security within the range, e.g., typically within 10 ft.

The following describes a suitable wireless protocol to enable communication between the mobile device 14 and network 16 to enable the mobile device 14 to transmit data to the server 18.

An suitable wireless protocol is the Global System for Mobile Communication (GSM) standard. The GSM standard can be used to provide a connection between the mobile device 14 and the server 18. The GSM standard connects the mobile device 14 to a local exchange server that forwards data to the Internet and ultimately to the server 18. The standard is generally separated into three layers, namely the physical layer, the link layer, and the message layer.

The physical layer can be summarized as follows. In GSM, the radio channels are based on a Time Division Multiple Access (TDMA) structure that is implemented on multiple frequency sub-bands. Two frequency bands are used by the GSM system: 890-915 MHz for the mobile device 14 to server 18 direction; and 935-960 MHz for the server 18 to mobile device 14 direction. These bands are divided into 124 pairs of carriers spaced 200 kHz apart. Each 200 kHz channel is segmented in time using a TDMA scheme consisting of 8 time slots. The TDMA scheme uses a gross bit rate of approximately 270 kb/s using a Gaussian Minimum Shift Keying (GMSK) modulation.

The link layer is used to link the server 18 to the mobile device 14. The link frame is typically made up of five fields: the address field, the control field, the length indicator field, and the fill-in bits field. The address field is used to carry the service access point identifier (identifier for the current mobile device 14). The control field is used to carry sequencing numbers and specification of the type of frame. The link layer uses three types of frames for supervisory functions, unnumbered control functions, and numbered multi-frame information transfer frames. The length indicator is used to distinguish the information-carrying field from the fill-in bits. The information field is used to carry the desired information. The fill-in bits field is used to fill the rest of the frame to the required length.

The message layer is used to link the mobile device 14 to the local exchange server. This layer is first used to establish signalling and traffic channels between the mobile device 14 and the server 18. Then the layer is used to establish a connection from the server 18 to the local exchange server. The layer is finally used to manage the connection from the mobile device 14 to the local exchange server. The local exchange server uses this information to forward all data to either the phone network or the Internet.

It will be appreciated that the above protocol is shown for illustrative purposes only, and that any suitable protocol for communicating between the mobile device 14 and the server 18 may be employed.

The mobile device 14 is responsible for transmitting sensor data to the server 18. In this example, by using a cell phone or PDA, the device 14 is already configured to access the network 14 directly (i.e., the cell phone or PDA is capable of directly accessing the Internet in this case). Therefore, the system 10 may employ whichever transmission scheme is used by the device 14, especially when it is a pre-existing device. However, if the device 14 is a custom manufactured device, the choice of transmission scheme may depend on the application, as well as other considerations, such as cost. In either situation, any suitable RF transmission scheme that can communicate with the network 16, such that sampled sensor data can be uploaded to the server 18 to be accessed by the interface software 62 and analysis software 60, may be used by the device 14.

The following will discuss steps 516 to 526, outlined in FIG. 4, in greater detail.

At step 516, the server 18 receives the sensor data. For the server 18 to “receive” data, the sensor data is uploaded to the server 18 through the network 16. The device's software module 40 organizes the sensor data into a format accepted by the network 16, such that the sensor data can be sent through the network 16 to the server 18. The analysis software 60 identifies the sensor data based on the identifier 32 unique to each sensor module 12. The software 60 uses the identifier 32 to correlate the sensor data with the particular patient's information.

At step 518, the analysis software processes the sensor data, and provides an output that is suitable for use. This processing of the data may produce any desired output, such as, graphical output, and numerical statistics. Therefore, the analysis software 60 may access patient information stored in the database 58, through the server 18, and the server 18 communicates sensor parameters to the analysis software 60 that need to be analysed from the sampled data. The analysed data is stored on the server 18. The server 18 is then able to provide the processed data, in a useable form, to, e.g., the observer 17 using the interface software 62 as indicated in step 520.

As the sensors 20 are monitoring the patient, and transmitting the sensor data to the server 18 through the device 14, the patient may also keep track of their vital signs by occasionally viewing the display 34 at step 512. The mobile device's software module 40 is therefore capable of processing the sampled data, in order to provide a useable output on the display 34. It shall be noted that this step is optional depending on the application of the system 10 and the nature of the device 14.

The continuous monitoring of a patient may be accomplished using the analysis software 60, wherein an algorithm is used to detect anomalous situations which may pose a threat to the patient. Monitoring may also be done by the observer 17 (i.e. a treatment provider in this example). Alternatively, a hybrid scheme may be employed, wherein an algorithm continuously monitors the patient's vitals while the observer 17 periodically analyzes the data and is alerted by the analysis software 60 if an emergency situation has been detected. This arrangement would be especially useful if the observer 17 and the server 18 are at the same location, such as a hospital. Therefore, monitoring a patient can be accomplished in any number of ways depending on the nature of the application, and the degree of urgency, which is required for responding to a patient's needs.

It is preferable to maintain a connection between the sensor module 12 and the device 14 to enable continuous monitoring. However, the transmission link between the device 14 and the server 18 can be intermittent due to wireless availability.

When the observer 17 wishes to access the sensor data, a secure connection with the interface software 62 would typically be required, since the patient's information contained in the server 18 and database 58, as well as the vital statistics themselves would be confidential. Therefore, the observer 17 would need to have been granted permission to access the information. It shall be noted that in other applications, such as for environmental monitoring, the data being displayed may not be confidential, in which case any observer 17 that has access to the network 16 may be allowed to view the sensor data.

A suitable procedure for accessing the sensor data through the interface software 62 will now be described, referring to FIGS. 5-11.

In order to establish a secure connection to the interface software 62, the observer 17 would first be required to login. A typical login window 110 is shown in FIG. 5. The login window 110 has a username entry box 112, a password entry box 114, and a login button 116. The observer 17 would input their username and password in the appropriate boxes, and select the login button 116.

The observer 17 would then need to access the appropriate data stored on the server 18, which has access to the database 58. This step may be optional. Alternatively, once the observer 17 logs in, they may have complete access to the database 58, through the server 18, which stores previous sensor data and patient information. Connection with the server 18 is done using a connection interface console 190, such as that shown in FIG. 10. The console 190 has folder input box 192, a port input box 194, a connect button 196, and a disconnect button 198. Upon entering the appropriate data into the boxes 192 and 194, the observer 17 may then connect to the server 18 by selecting the connect button 196. To disconnect from the server 18, the observer 17 may do so by selecting the disconnect button 198.

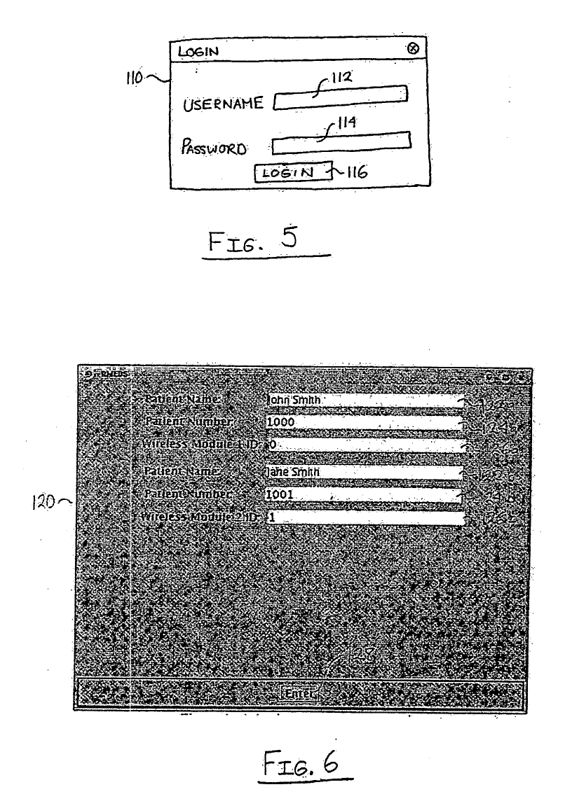

It will be assumed for this example that the observer 17 wishes to monitor two patients, namely John Smith, and Jane Smith. Therefore, in this case, data from two sensor modules 12 will be monitored. In order to monitor John and Jane, the observer 17 would be required to enter patient information through a data entry interface 120, shown in FIG. 6. A name 122a, patient number 124a, and sensor module ID 126a are input by the observer 17 for John and a similar set of fields, 122b, 124b, and 126b, are populated to enter Jane's information. The observer 17 would then select an enter button 128 to enter John and Jane's information.

After completing the above data entry, John and Jane's sensor modules 12 are correlated with John and Jane's personal information stored in the database 58 using the analysis software 60 and stored on the server 18. The data is then available to the interface software 62. The interface software 62 provides the observer 17 with a main interface console 130, such as that shown in FIG. 7. The console 130 has an error box 132 for displaying errors related to John's sensor module, and an error box 134 for displaying errors related to Jane's sensor module. The console 130 also has a refresh button 136, to allow the observer 17 to refresh the console 130, and a button for each sensor module 12, denoted by numerals 136 and 138 respectively.

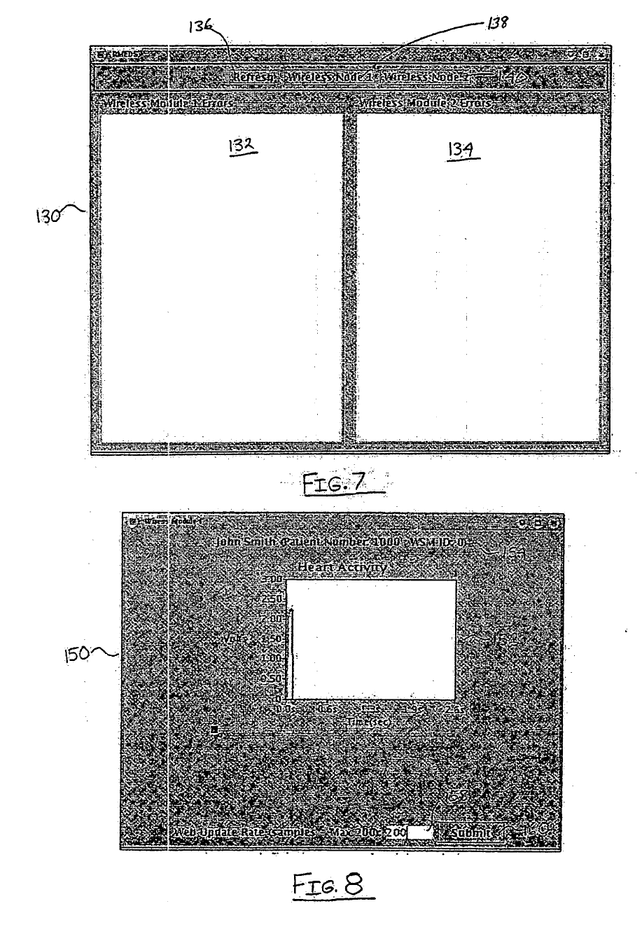

By selecting the appropriate sensor module button (i.e. 136 or 138), the observer 17 can open an interface which displays the sensor data in a useable form. An example is shown in FIG. 8. The graphical interface 150 shown in FIG. 8 corresponds to John's sensor module. To open this window, the observer 17 would select the button 136, which corresponds to John's sensor module. The interface 150 has an identification label 154, showing the patient's name, number and sensor module ID number; a graphical output 152, showing the patient's vital sign(s) (in this case John's heart activity); an entry box 158 to allow the observer 17 to change the update rate of the data; and a submit button 160 to allow the observer 17 to change the update rate. Any changes to the update rate are stored on the server 18 and communicated to the mobile device 14 as mentioned above.

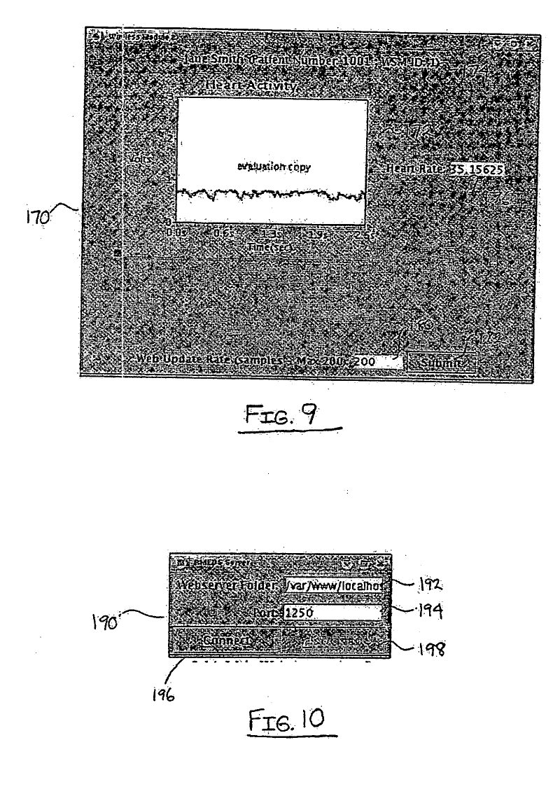

FIG. 9 shows a graphical interface 170 corresponding to Jane's sensor data. The interface 170 has an identification label 174; a graphical output 172; an entry box 180; and a submit button 182 similar to John's interface 150. However, the interface 170 also provides a digital readout 176 of Jane's heart rate. Therefore, the features shown in the interfaces 150 and 170 are for illustrative purposes only, and it will be appreciated that any number of features may be provided. For instance, the observer 17 may wish to have access to the patient's medical records. This may be included into the interface 150 or 170 to permit such access in a convenient manner.

Access to a patient's medical records is preferable in a medical application, since the observer 17 can compare the vital signs shown in 152 or 172, with the patient's records, enabling a more accurate diagnosis. This may prevent, e.g., an improper dosage of medication, should the patient require specific medication to balance the particular vital sign(s). The observer 17 may be able to assess anomalous situations more quickly, since previous diagnoses can assist with such determinations. Moreover, the observer 17 is able to know the particular medication being used by the patient, which helps to minimize the administration of conflicting medications.

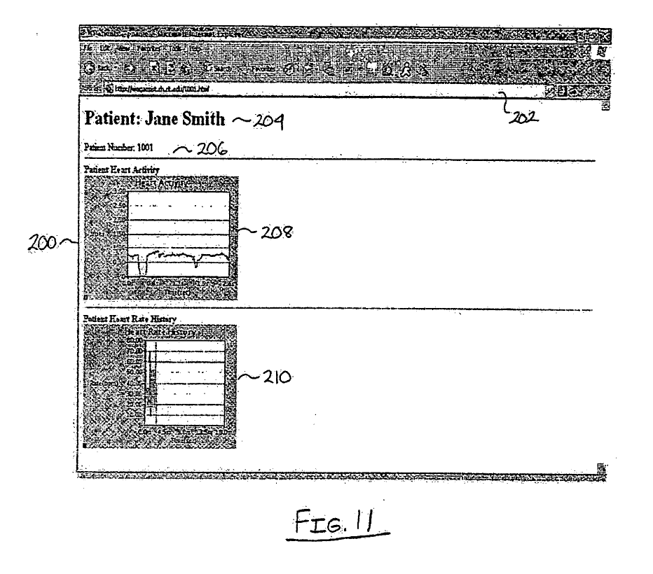

A monitoring interface 200 provided by the interface software 62, is shown in FIG. 11. Preferably, when the network 16 is the Internet, the interface 200 is a standard web-browser. The interface 200 has an address toolbar 202, an identifier 204, patient number 206, a heart rate activity graphical output 208, and a heart rate histoiy graphical output 210. An interface such as 200 is also a suitable interface to be used by the software module 40, for displaying the sensor data in a useable form on the display 34 of the device 14 (i.e. in step 512). However, the interface 200 is most applicable for enabling a observer 17 to view the patient's vitals during normal monitoring. The interface 200 preferably has an automatic refresh rate to enable the interface 200 to display an output substantially in real-time.

Therefore, once the observer 17 has provided the appropriate inputs to the interfaces 110, 120, 150 and 170; the observer 17 may then commence a typical monitoring procedure using the interface 200. Since the data is provided through interface software 62 provided by the server 18, multiple observers can access the data, and the data can be accessed from any location which has a connection to the network 16 or directly to the interface software 62 or server 18.

The preferred embodiment of the present invention provides a portable, modular system 10, that allows a patient and an observer 17 to monitor particular vital signs. The patient wears a sensor module 12 that is capable of sampling data from a series of sensors and wirelessly transmitting the data to a mobile device 14. The patient can be carrying the device 14 or the device 14 can be placed nearby the patient. The device 14 is portable, can have a display 34, and can communicate wirelessly with both the sensor module 12 and the network 16. This enables the device 14 to not only transmit sensor data that it has received from the sensor module 12, to a server 18 to be processed by analysis software 60; but also to have itself process and display the sensor data in a useable form for the patient to view their vital signs. The device 14 also enables emergency personnel 19 and the analysis software 60 to contact the patient directly, should an emergency arise.

The device's software module 40 is capable of processing the sensor data that has been sampled from the sensor module 12, enabling the sensor data to be presented in a graphical output on a display 34, enabling the patient to continuously monitor the particular vital sign(s) of interest, An observer 17 can monitor the vital signs through the interface software 62. The interface software 62 provides a number of user interfaces, and can restrict access to the server 18 which contains patient information and archived sensor data. Thus, an observer 17 that wishes to access the server 18 through the interface software 62 to monitor a patient, requires the proper authorization to do so.

It will be appreciated that the above-described interfaces are shown for illustrative purposes only, and that any suitable interfaces or interface software 62 may be implemented, as required by the specific application. For example, the interfaces may form an integral console, which provides all of the above features in a single window. The output viewed by a patient on the display 34 may also take any suitable form, depending on the capabilities of the software module 40 and processor 38.

It will also be appreciated that the system 10 may be used for applications other than those that are medical related. For example, the sensor module 12 may have sensors 20 that are configured to measure environmental parameters such as the carbon monoxide level in a building. in such an application, the basic structure of the system as outlined in FIG. 1 would be applicable, however, the wireless protocol may be adapted to suit the number of sensors or number of modules, and the interfaces provided by the interface software 62 and shown in FIGS. 5-11 may be modified or simplified. For instance, the software interface 62 may not require secure access in this scenario, and may be greatly simplified to run entirely alongside the server 18. If an emergency is detected, an alarm, e.g., can be initiated to notify the proper authorities.

Therefore, the system 10 provides a highly effective monitoring system that is modular, expandable and adaptable to any environment or situation employing sensors and that requires monitoring. Particularly, the use of a mobile device 14, preferably a cell phone or PDA allows complete portability. Not only are the sensors modular, the device 14 is modular, and thus can be, e.g., carried around with a patient; or placed in a remote, dangerous location, that may be inaccessible to a human. The display 34 also provides to the user of the sensor module 12, the opportunity to receive substantially immediate feedback on the parameters being monitored; and if the telephone module 46 is used, the user can be contacted directly, or alternatively, they may contact the emergency personnel 19 themselves.

It will be appreciated that the telephonic functionality of the device 14 may be replaced by a module that supports another communication medium, such as email. This would enable the user of the sensor module 12, and the server 18, to maintain contact therebetween by sending electronic messages. The incorporation of a telephone module 46 is entirely optional, especially when a custom device 14 is used, and the particular situation does not require auditory alerts.

The use of a cell phone or PDA is also particularly beneficial due to their relatively long battery life, and the fact that they are rechargeable. If the system's application requires continuous monitoring, it is beneficial to have a strong battery to avoid the need to frequently close to a static power source.

The modularity of the system 10 also provides relatively simple implementation, since the device 14 may be a pre-existing unit, requiring only a software program; the server is primarily software based, which can be run from any PC; and a network such as the Internet comprises pre-existing infrastructure. The sensor modules 12 are preferably compact, lightweight devices, and therefore can be manufactured with any desired sophistication to suit the cost requirements for the particular application.

Although the invention has been described with reference to certain specific embodiments, various modifications thereof will be apparent to those skilled in the art without departing from the spirit and scope of the invention as outlined in the claims appended hereto.

What is claimed is:

1. A sensor system for monitoring sensor data comprising:
a sensor module having a sensor and a first wireless link that wirelessly transmits data sampled from the sensor to a mobile communication device;
the mobile communication device having a second wireless link that receives the data from the sensor module and wirelessly transmits the data to a server.

2. The sensor system according to claim 1 wherein the server has a computer program that processes the data into a useable form and uploads the data in its useable form onto a network.

3. The sensor system according to claim 1 wherein the sensor module is used for monitoring physiological signals and is worn by a patient.

4. The sensor system according to claim 1 wherein the data is accessible by a monitoring entity via an interface provided by the computer program.

5. The sensor system according to claim 1 wherein the data is accessible via the mobile communication device.

6. The sensor system according to claim 1 wherein a two-way communication channel is provided between the server and the mobile communication device.

7. The sensor system according to claim 1 wherein the mobile communication devices receives data from a plurality of sensor modules.

8. The sensor system according to claim 7 wherein each sensor module has a unique associated identifier associated with the data from each sensor module and the identifier is accessible by the server.

9. The sensor system according to claim 1 wherein the sensor module has a plurality of sensors.

10. The sensor system according to claim 1 wherein the first wireless link is a short-range wireless link.

11. The sensor system according to claim 1 wherein the first wireless link comprises a wireless communication protocol chosen from the group of: a radio frequency communication protocol, a magnetic induction protocol, and a wireless personal area network protocol.

12. The sensor system according to claim 1 wherein the second wireless link comprises a wireless communication protocol chosen from the group of: GSM standard, GPRS, GPS, 3G, WIFI (801.11), WiMAX, and a radio frequency communication protocol.

13. The sensor system according to claim 1 wherein the mobile communication device is chosen from the group of: a cellular phone, a personal digital assistant, and a portable computer.

14. The sensor system according to claim 1 further comprising a database storing sensor module-specific information and accessible by the server.

15. The sensor system according to claim 2 wherein the network is chosen from the group of the Internet and an internal network.

16. A method for acquiring and monitoring sensor data comprising the steps of:
sampling data from a sensor controlled by a sensor module;
the sensor module wirelessly transmitting the data to a mobile communication device;
the mobile communication device wirelessly uploading the data to a server connected to a computer program, the server providing a two-way communication channel between a network and the mobile communication device;
the computer program processing the data into a useable form; and
the computer program providing the data in its useable form over the network to be accessed by a monitoring entity.

17. The method according to claim 16 wherein the sensor is worn by a patient, further comprising the step of the computer program analyzing the data to detect an emergency and in the case of an emergency, contacting the monitoring entity through the network and contacting the patient through the mobile communication device.

18. The method according to claim 16 further comprising the step of the computer program providing the data to the monitoring entity through a computer interface, and the monitoring entity controlling the operation of the sensor through the network.

19. The method according to claim 16 further comprising the step of the mobile communication device providing an observer with access to the data.

20. The method according to claim 16 wherein the sensor module wirelessly transmits the data using a wireless communication protocol chosen from the group of: a radio frequency communication protocol, a magnetic induction protocol, and a wireless personal area network protocol.

21. The method according to claim 16 wherein the mobile communication device and wirelessly uploads the data using a wireless communication protocol chosen from the group of: GSM standard, GPRS, GPS, 3G, and a radio frequency communication protocol.

22. The method according to claim 16 wherein the mobile communication device is chosen from the group of: a cellular phone, a personal digital assistant, and a portable computer.

23. The method according to claim 16 further comprising the step of matching the data to sensor module-specific information stored in a database.

24. The method according to claim 16 further comprising the step of updating the mobile communication device with the data in its useable form.
